(19) United States
(12) Patent Application Publication
(10) Pub. No.: US 2006/0282161 A1
(43) Pub. Date: Dec. 14, 2006

(54) Valve annulus reduction system

(75) Inventors: Rany Huynh, Charleston, MA (US); Eliot Bloom, Hopkinton, NH (US); Jack Lemmon, Saint Paul, MN (US); Timothy R. Ryan, Shorewood, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 10/561,113

(22) PCT Filed: Jun. 21, 2004
(86) PCT No.: PCT/US04/19814

§ 371 Date: Jun. 5, 2006

Related U.S. Application Data
(60) Provisional application No. 60/480,201, filed on Jun. 20, 2003.

(51) Int. Cl. A61F 2/24

(52) U.S. Cl. 623/2.11; 600/37; 623/2.37

(57) ABSTRACT

System (10) for treating a dilated heart valve includes a dilivery device. Tension device (12) is slidably received within the lumen of delivery device (14) for delivery to and deployment at the treatment area. Delivery device (14) includes a plurality of arranged catheters including an inner catheter (26) within the lumen of an outer catheter (22). Tension device (12) includes a first anchor (16) attached to a second anchor (18).

### RELATED APPLICATION

This application claims priority to U.S. Provisional Application No. 60/480,201, “Coronary Sinus Approach for Repair of Mitral Valve Insufficiency” to Rany Huynh, et al., filed Jun. 20, 2003, the entirety of which is incorporated by reference.

### TECHNICAL FIELD

The technical field of this disclosure is medical devices, particularly, for reducing a valve annulus.

### BACKGROUND OF THE INVENTION

Valve insufficiency is a potentially grave health issue that can lead to cardiac dysfunction. Mitral valve insufficiency may comprise a valve that does not completely shut and affect the seal between the left ventricle and the left atrium. Historically, such a condition necessitated surgical intervention.

Surgical repair of mitral valve insufficiency involved the use of a sternotomy or a similar invasive procedure. After performing a sternotomy, the patients heart would be stopped while the surgeon transected the chambers of the heart to gain access to the mitral valve. Upon attaining access to the mitral valve, the surgeon could then repair the valve by an annuloplasty, or suturing the valve. These procedures are complex, time consuming, and involve many risks attendant with open cardiac surgery. Complications may occur, and recovery time may be significant.

Catheter based valve replacement has been proposed as a way to effect valve replacement percutaneously and to avoid open-heart surgery. Such procedures involve excision of the native valve and replacement of the native valve with a prosthetic valve, or installation of a prosthetic valve over the native valve, or installation of a device on or adjacent the valve to repair the damaged valve. Previous proposed treatments also involve the use of clips to bind the posterior and anterior leaflets of the mitral valve. To avoid cardiopulmonary bypass, the catheter based valve replacement is performed on a beating heart. Following excision of the native valve, no valve is present to preserve the pumping action of the heart while the permanent prosthetic valve is being implanted.

An additional consideration in both open-heart and catheter based valve replacement is the healing process after the prosthetic valve is implanted. After the surgical valve replacement procedure, scar tissue must form around the sewing cuff to secure the prosthetic valve in position. In current practice, multiple knotted sutures anchor the prosthetic valve in place until ingrowth of scar tissue into the sewing cuff takes over the load bearing function. The placement of knotted sutures by catheter can be very difficult and time consuming.

Artificial heart valves for temporary use are known in the art, but present certain problems. Some designs are complex, requiring inflating and deflating balloons to alternately block and permit flow. Such designs require complex sensing and control systems. Other designs fail to provide access for tools that must reach the valve site for removal of the native valve and placement of the prosthetic valve. Yet other designs require elaborate supporting frames to hold the valve portion.

U.S. Pat. No. 3,671,979 to Moulopoulos discloses an artificial heart valve for implantation in close proximity to a malfunctioning or damaged natural aortic or mitral heart valve by remote means without performing an open chest or other major surgical operation, the artificial heart valve comprising a flexible membrane in the form of an umbrella.

U.S. Pat. No. 4,056,854 to Boretos et al. discloses an artificial valve remotely placeable in a blood vessel without major surgery to supplant the function of a malfunctioning natural valve including an expansible check valve remotely placed in a constricted configuration through the vessel and a remotely removable constraint for selective expansion of the check valve for sealing engagement thereof within the walls of the vessel at the desired location.

U.S. Pat. No. 4,705,507 to Boyles discloses an arterial catheter of the multi-lumen type having an inflatable balloon portion to wedge the catheter in place against the arterial wall. Multi-infusions are allowed through the segmented multi-lumens. The catheter is designed to allow blood to flow in the arterial system with the catheter in place. During diastolic phases, the blood flow will be closed off with movable plastic valves.

U.S. patent application Ser. No. 20020151970 to Garrison et al. discloses a valve implantation system having a valve displacer for displacing and holding the native valve leaflets open wherein a replacement valve may be attached to the valve displacer before or after introduction and may be positioned independent of the valve displacer and wherein a temporary valve mechanism may be used to provide temporary valve functions during and after deployment of the valve displacer.

WIPO International Publication No. WO 00/44313 to Lambrecht et al. discloses temporary valve devices with one or more cannulae that guide insertion of the valve into the aorta. The valve devices expand in the aorta to occupy the entire flow path of the vessel. In one embodiment, the temporary valve has leaflets that act in concert to alternately block or allow blood flow.

Another approach to repair of mitral valve insufficiency is reducing the size of the annulus. Prior art attempts to reduce the annulus provide a tension device anchoring outside two cardiac walls, with spherical anchors. The prior art solutions incur potentially undesirable trauma to the cardiac tissue. Furthermore, the prior art spherical anchors concentrate the strain provided by the tension over a relatively small surface area of the cardiac tissue. Examples of these devices are disclosed in U.S. Pat. No. 6,332,893 to Mortier, et al, U.S. Pat. No. 6,261,222 to Schweich, et al and U.S. Pat. No. 6,260,552 to Mortier et al.

It would be desirable therefore to provide an apparatus and method that overcomes these, and other, problems.

### SUMMARY OF THE INVENTION

The invention provides a device for treating a dilated cardiac valve. The device comprises a first anchor disposed on a first end of a tension member and a second anchor slidably mounted on a second end of the tension member. The second anchor has an arcuate tubular body that complements a curve of at least a portion of a cardiac vessel adjacent the dilated valve.

The invention also provides a system for treating a dilated heart valve. The system includes a tension member connected by first and second anchors, and a telescoping set of catheters to deliver the tension member to a position adjacent the dilated heart valve

The invention further provides a method for treating a dilated heart valve comprising delivering a tension device comprising a barbed anchor connected to a radiused anchor with a cord to a location within an atrium proximal the dilated heart valve. The method further provides for inserting the barbed anchor into a first atrial wall proximal the dilated heart valve and positioning the radiused anchor inside the coronary sinus opposite the first atrial wall. The method then reduces an annulus of the dilated heart valve via the tension device.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE FIGURES

FIG. 1 illustrates one embodiment of a system for treating a dilated heart valve in accordance with the present invention.

FIGS. 2 to 6 illustrates various views of one embodiment of a proximal anchor used in the system illustrated in FIG. 1.

FIGS. 7-9 illustrate other embodiments of proximal anchors that may be used in the system illustrated in FIG. 1.

FIGS. 10 and 11 illustrate one embodiment of a locking mechanism used in the system illustrated in FIG. 1.

FIGS. 12 and 13 illustrate another embodiment of a locking mechanism used in the system illustrated in FIG. 1.

FIGS. 14 and 15 illustrate another embodiment of a locking mechanism used in the system illustrated in FIG. 1.

FIG. 16 illustrates a delivery device positioned adjacent a heart valve in accordance with an aspect of the invention.

FIG. 17 is a flowchart illustrating an exemplary method for treating a dilated heart in accordance with another aspect of the invention.

### DETAILED DESCRIPTION OF THE PRESENTLY PREFERRED EMBODIMENTS

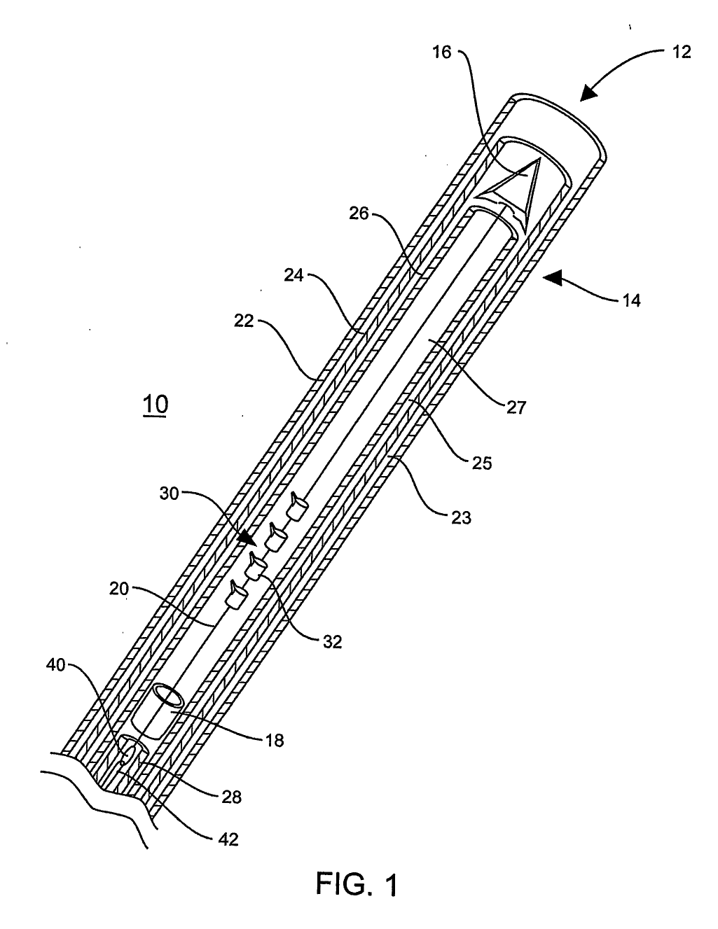

One aspect of the present invention is a system for treating a dilated heart valve. The system may be used to treat any one of the cardiac valves. The description below provides detail for treating the mitral valve via a catheter routed through the coronary sinus. Alternative embodiments may treat mitral or tricuspid valves using a tension device delivered via a catheter through a coronary vein or artery and into a chamber of a heart. The coronary vessels used for accessing a heart chamber may lie in either a septal wall or an outer, free wall of the heart. The heart chamber may be an atrium or a ventricle. The tension device is routed from the coronary vessel into the adjacent chamber of the heart. A distal anchor of the tension device is embedded in an opposing chamber wall and a proximal anchor of the tension device is deployed in the coronary vessel. Applying tension to the tension device will shorten the length thereof, thus reducing the dilated annulus of an adjacent cardiac valve. One embodiment of the system, in accordance with the present invention, is illustrated in FIG. 1.

Referring to FIG. 1, one embodiment of a treatment system for dilated heart valves is generally shown at numeral 10. The treatment system includes a tension device 12, a delivery device 14 and a locking mechanism 30. Delivery device 14 comprises a plurality of concentrically arranged catheters. In another embodiment, delivery device 14 comprises an inner catheter disposed within lumen of an outer catheter. In yet another embodiment, delivery device 14 comprises a plurality of catheters sequentially delivered to a delivery site. In another embodiment for use during surgical techniques, the delivery device may be a trocar or a cannula. Alternatively, a minimally invasive approach employing an endoscope may be used. Delivery device 14 illustrated in FIG. 1 is discussed in more detail, below.

Tension device 12 includes first (distal) anchor 16 attached to second (proximal) anchor 18 with tension member (tether) 20. Tension device 12 is axially disposed within lumen 27 of inner catheter 26 during delivery. As used herein, the terms “distal” and “proximal” are with reference to the treating clinician during deployment of the device: “Distal” indicates a portion distant from, or a direction away from the clinician and “proximal” indicates a portion near to, or a direction towards the clinician.

Tension member 20 is composed of a biocompatible material having sufficient tensile strength for maintaining an applied tension. In one embodiment, tension member 20 comprises a biocompatible metallic or polymeric material that combines flexibility, high strength, and high fatigue resistance. For example, tension member 20 may be formed using materials such as stainless steel, titanium, a nickel-titanium alloy, a nickel-cobalt alloy, another cobalt alloy, polypropylene, polyethylene, polyurethane, polytetrafluoroethylene (PTFE), polyester (Dacron® polyester), nylon, combinations thereof, and the like. In one embodiment, tension member 20 may comprise a polymeric filament having an elastic property that decreases linearly or in an abrupt step when a desired tether length is reached. In one embodiment, tension member 20 is a predetermined length.

In one embodiment, an antithrombotic component may be included in the chemical composition of a polymeric filament tension member. Alternatively, a polymeric or metallic tether may be coated with a polymer that releases an anticoagulant and thereby reduces the risk of thrombus formation. If desired, additional therapeutic agents or combinations of agents may be used, including antibiotics and anti-inflammatories.

First anchor 16 is fixedly fastened adjacent a distal end of tension member 20. The first anchor 16 is fashioned to be inserted into a cardiac wall such as a valve annulus or a septum adjacent thereto. The first anchor 16 may be a hooked anchor, a coil barbed anchor, a spiral anchor or pigtail shaped anchor or a harpoon shaped device. The first anchor 16 is composed of a biocompatible material. The first anchor 16 can be made of stainless steel, nitinol, tantalum, MP35N cobalt alloy, platinum, titanium, a thermoset plastic, or a combination thereof.

Second anchor 18 is slidably mounted about and lockable to a proximal end of tension member 20, as will be described in more detail below. Second anchor 18 includes an arcuate length that conforms to the curvature of the coronary sinus. Second anchor 18 may include a radius that conforms to at least a portion of the radius of the circular transverse cross section of the lumen of the coronary sinus. Second anchor 18 is composed of a biocompatible material. Second anchor 18 can be made of, for example, flexible stainless steel, nitinol, biocompatible durable shape-memory polymers, cobalt-based alloys, such as MP35N, or a combination thereof.

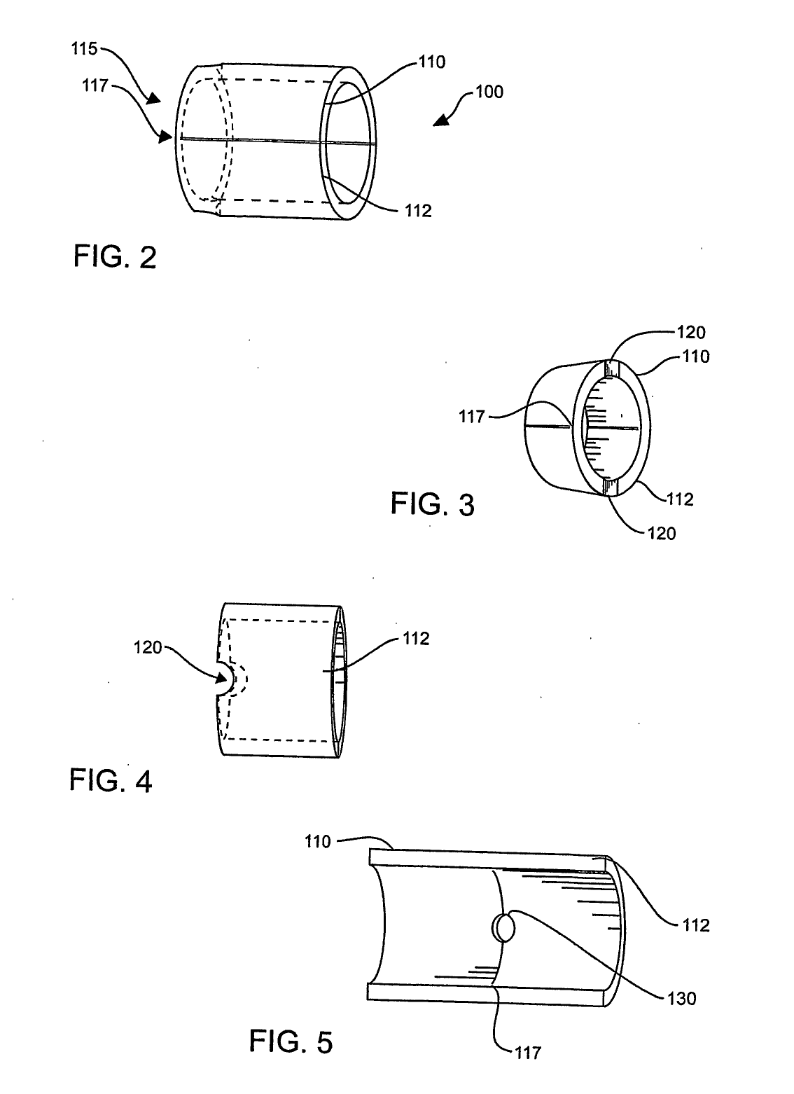

FIGS. 2 through 6 illustrates one embodiment of the second anchor 18 of system 10 illustrated in FIG. 1, the embodiment referred to generally as second anchor 100. Second anchor 100 is delivered to the coronary sinus in a tubular delivery state illustrated in FIGS. 2-4 and opened within the coronary sinus to form a treatment state illustrated in FIG. 5. Second anchor 100 is composed of a hollow arcuate tube cut along a longitudinal axis to form a first anchor portion 110 and a second anchor portion 112 each having a generally C-shaped cross section. Anchor portions 110, 112 are connected at end 115 by hinge 117 as best seen in FIG. 5. Hinge 117 may be a spring hinge that opens anchor 100 into the treatment state when anchor 100 is released from inner catheter 26 of system 10 illustrated in FIG. 1. Anchor portions 110, 112 include notches 120, 122 at end 115 of anchor 100. As shown in FIG. 3, notch 120 is positioned opposite notch 122 when anchor 100 is in the delivery state. Notch 120 and notch 122 form opening 130 when anchor 100 is opened into the treatment state illustrated in FIG. 5. Opening 130 provides passage for tether 20 during placement of tensioning device 12 of system 10. FIG. 6 illustrates, on the left side, anchor 100 in the closed delivery state and, on the right side, the open treatment state. In the embodiment shown, anchor 100 opens in the direction of arrow A when deployed within the coronary sinus.

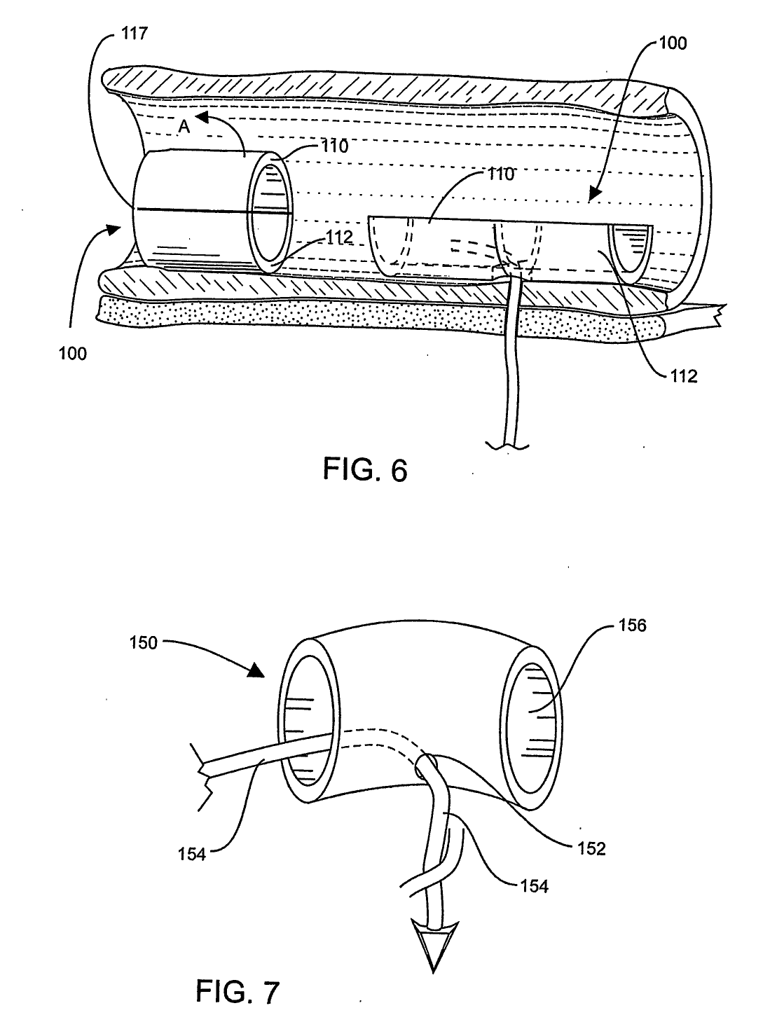

FIG. 7 illustrates another embodiment of second anchor 18, referred to generally as second anchor 150. Second anchor 150 has a radius that conforms to the radius of at least a portion of the coronary sinus adjacent the posterior leaflet of the mitral valve. Second anchor 150 is formed from a short section of tubing having a circular cross section and an outer diameter that is less than the inside diameter of the coronary sinus. In the embodiment illustrated in FIG. 7, second anchor 150 is formed from an arcuate tube. Second anchor 150 includes a side opening 152. Side opening 152 provides a passage for tether 154 into and through lumen 156 of second anchor 150. Second anchor 150 may be composed of material similar to those materials discussed above for anchor 18 illustrated in FIG. 1.

FIG. 8 illustrates yet another embodiment of second anchor 18, referred to generally as second anchor 200. Second anchor 200 has an arcuate length that conforms to the radius of curvature of at least a portion of the coronary sinus adjacent the posterior leaflet of the mitral valve. Second anchor 200 comprises an open channel 216 having a generally C-shaped cross section. In one embodiment, second anchor 200 is laser cut from a tubular body. Second anchor 200 includes a side opening 212. Side opening 212 provides a passage for tether 214. Second anchor 200 may be composed of material similar to those materials discussed above for anchor 18 illustrated in FIG. 1.

In one embodiment, second anchor 18 of system 10 may comprise a self-expanding stent or a balloon-expanding stent. FIG. 9 illustrates another embodiment of second anchor 18, referred to generally as second anchor 250. Second anchor 250 is composed of a stent-like member 252 having a sidewall portion that has a transverse radius that conforms to the inside diameter of the lumen of the coronary sinus. Additionally, stent-like member 252 is formed to complement a curvature of the coronary sinus wall. Tether 254 may pass through any one of a plurality of openings defined by two adjacent struts of stent-like member 252. Second anchor 250 is composed of material similar to those described above for second anchor 18 or any other material well known in the art suitable for forming stents or stent-like structures.

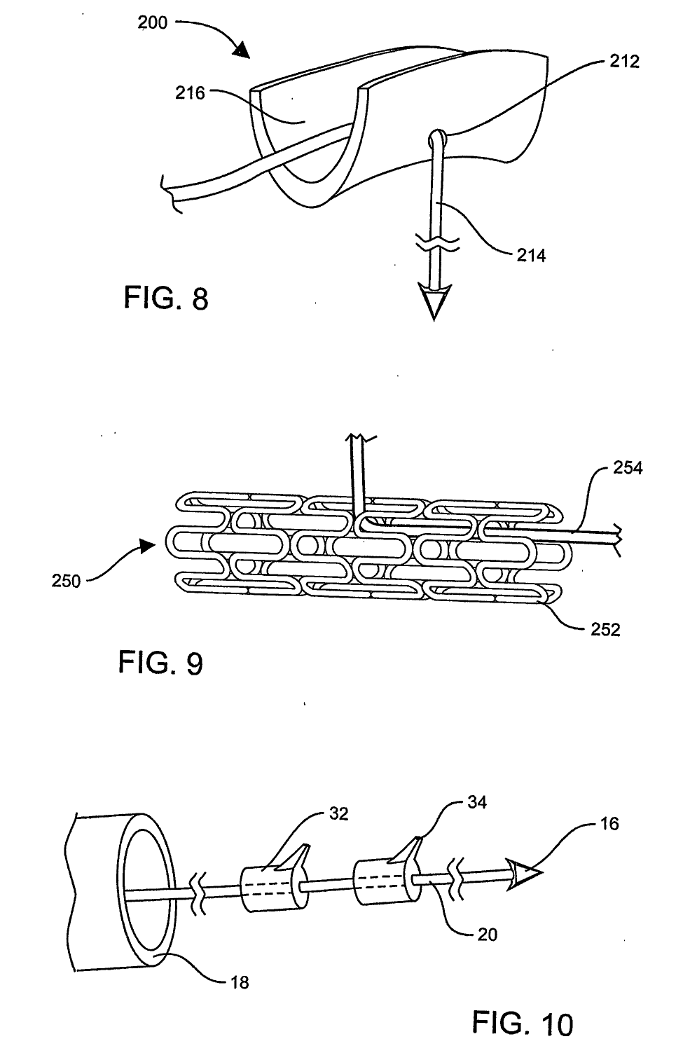

FIG. 1 illustrates that proximal second anchor 18 is variably attached to tether 20 by a locking member 30 affixed to tether 20. FIGS. 10 and 11 illustrate one embodiment of locking mechanism 30 shown in FIG. 1.

Locking mechanism 30 includes a plurality of locking members 32. At least one locking member 32 of locking mechanism 30 is drawn from an initial position between anchors 16, 18 to a position proximal the proximal second anchor 18. This not only locks the proximal second anchor 18 onto the tether 20, but also adjusts the length of the tether to change the proximity of the anchors 16, 18 one to the other.

In the present embodiment, multiple locking members 32 are spaced apart on tether 20 between distally positioned first anchor 16 and proximally positioned second anchor 18, affixed by, for example, crimping or swaging the locking members 32 onto the tether 20, confining each locking member with a knot or other enlargement on either side of the locking member, or using an adhesive. The length of the tether 20 between anchors 16, 18 is adjusted and maintained at the chosen length by drawing an appropriate number of locking members 32 through an opening of second anchor 18.

As shown in FIGS. 10-11, locking members 32 are formed of short sections of tubing having an outer diameter selected to provide a close sliding fit with an opening 36, such as openings 130, 152, 212 described above, of second anchor 18 (100, 150, 200, 250). Each locking member 32 includes flexible tab 34 flaring out at an angle from the longitudinal axis of the locking member. Tab 34 extends from the distal end of locking member 32 and flares out at approximately a 45-degree angle. Locking member 34 comprises a spring-like or shape-memory material. Tab 34 is heat set or otherwise set into its flared position.

As locking member 32 is drawn through opening 36 of second anchor 18, its tab 34 is bent back into alignment with the body of locking member 32 in order to fit through the opening. Once locking member 32 is no longer constrained by opening 36 of second anchor 18, tab 34 resumes its preset shape. The flaring tab 34 prevents locking member 32 from passing back through the second anchor 18, thereby locking second anchor 18 onto tether 20.

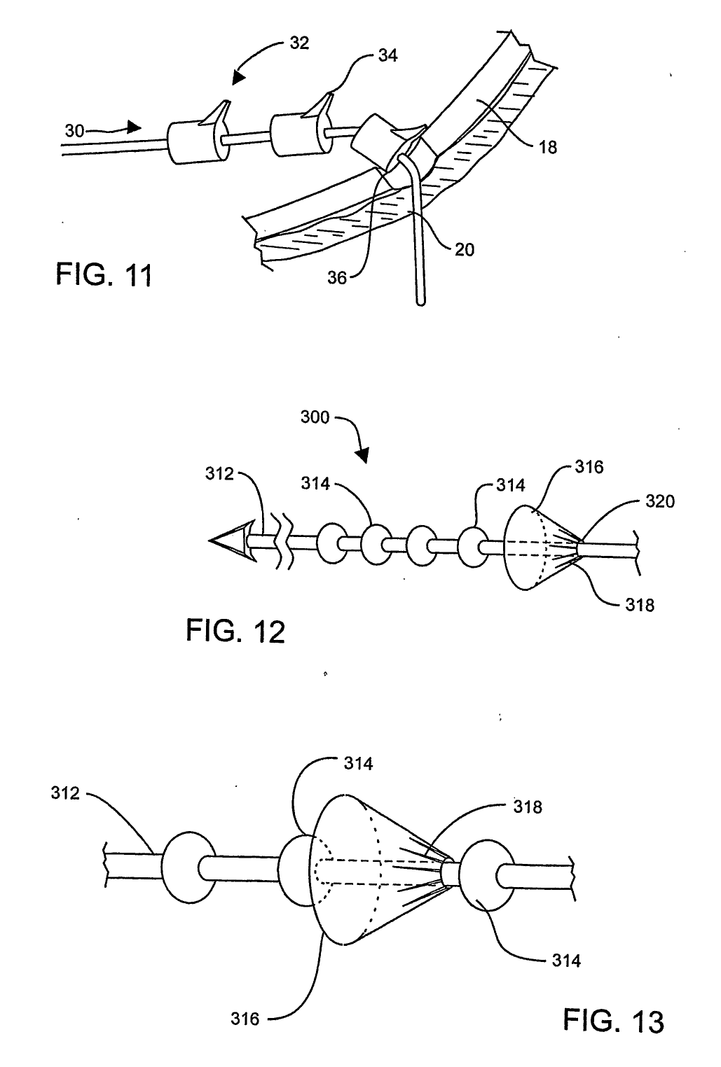

Any mechanism allowing tether motion in a proximal direction and preventing tether motion in a distal direction is suitable for the locking member. For example, FIGS. 12 and 13 illustrate another embodiment of a locking mechanism 30 suitable for use in system 10 and referred to generally as locking mechanism 300. Locking mechanism 300 includes a plurality of spherical locking members 314 disposed on tether 312. Locking mechanism 300 also includes a cone-shaped retaining device 316 having proximal opening 320 that allows passage of tether 312. In one embodiment, opening 320 has an inner diameter that is slightly less than or equal to the outer diameter of tether 312. Retaining device 316 includes at least one slit 318 proximate opening 320 that allows opening 320 to expand when a locking member 314 is drawn through opening 320. Retaining device 316 may be composed of any flexible material that allows opening 320 to expand as locking member 314 is drawn through cone 316 in a proximal direction and to return to the unexpanded state after the locking member 314 passes. In use, cone 316 is placed proximal to opening 36 of second anchor 18 and, in one embodiment, may rest against the second anchor.

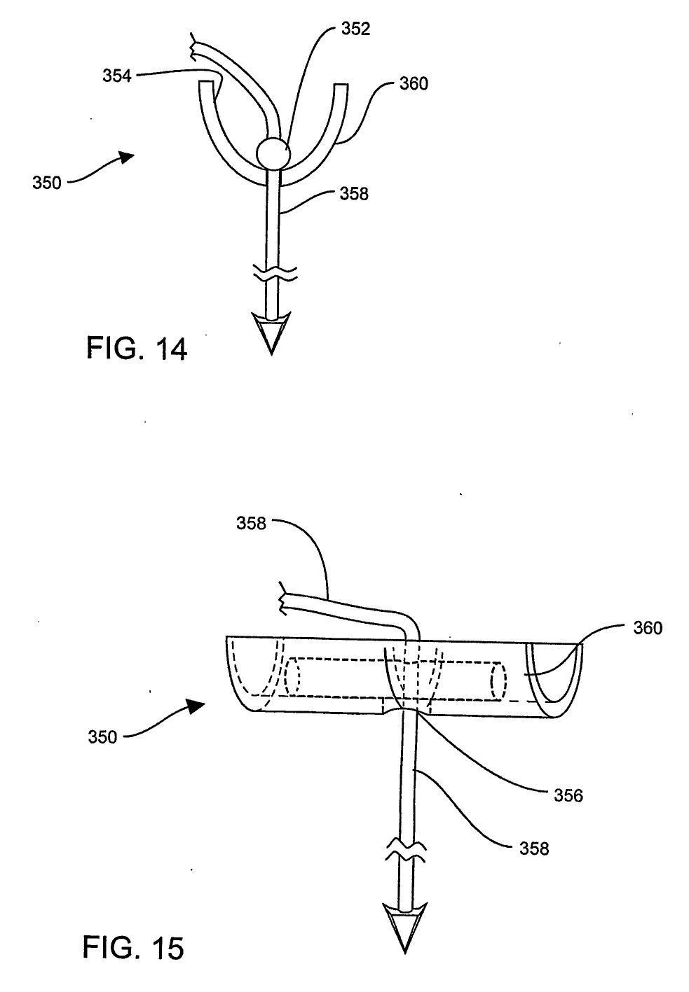

FIGS. 14 and 15 illustrate another locking mechanism 30 particularly suitable for use with hinged second anchor 100 illustrated in FIGS. 2-6, and referred to generally as locking mechanism 350. Locking mechanism 350 comprises rod 352 disposed within open channel 354 of the treatment state of hinged anchor 360. Rod 352 is sized to extend on either side of tether opening 356 and is composed of a rigid material suitable for preventing the movement of anchor 360 from the open treatment state to the closed delivery state. Rod 352 includes an opening (not shown) for passage of tether 358. Once the desired tension is placed, rod 352 may be secured to tether 358 by crimping the rod to the tether.

Returning to FIG. 1, tether 20 includes loop 40 on the proximal end of the tether. A length of suture material or another strong, thin, filament 42 passes through loop 40. The filament is roughly doubled over onto itself with the ends of the filament adjacent to each other and two portions of the filament extending away from loop 40. The filament is sized such that the ends of the filament extend outside the patient when tension device 12 is positioned at the treatment site. A treating clinician pulls both ends of the filament simultaneously to draw the appropriate number of locking members 32 through second anchor 18. Once the length of tether 20 has been adjusted and second anchor 18 has been locked onto tether 20, filament 42 is removed by releasing one end of the filament and pulling on the other end until the filament is withdrawn from the patient. This design eliminates the need to thermally cut or otherwise sever tether 20 after tension device 12 has been deployed at the treatment site.

As discussed above, system 10 for treating a dilated heart valve illustrated in FIG. 1 includes delivery device 14. Tension device 12 is slidably received within lumen of delivery device 14 for delivery to and deployment at the treatment area. As best seen in FIG. 1, delivery device 14 comprises outer catheter 22, delivery catheter 24, inner catheter 26 and holding tube 28. Delivery catheter 24 is slidable within lumen 23 of outer catheter 22, inner catheter 26 is slidable within lumen 25 of delivery catheter 24 and holding tube 28 is slidable within lumen 27 of inner catheter 26. Thus, delivery device 14 comprises four separate, concentric members, each slidable to be individually extended or retracted as needed to deliver tension device 12.

Outer catheter 22 comprises a flexible, biocompatible material such as polyurethane, polyethylene, nylon, or polytetrafluoroethylene (PTFE) or combinations of these materials. Outer catheter 22 may have a preformed or steerable distal tip that is capable of assuming a desired bend with respect to the longitudinal axis of the sheath, for example, a bend suitable for intubating the coronary sinus.

Delivery catheter 24 comprises the same or a different biocompatible material from that used to form outer catheter 22. Delivery catheter 24 must be flexible enough to be delivered through vasculature to the treatment area while still rigid enough to span the atrial chamber for delivering the first anchor for implanting into the septal wall.

Inner catheter 26 comprises the same or a different biocompatible material from that used to form outer catheter 22. Delivery catheter 24 must be flexible enough to be delivered through vasculature to the treatment area while still longitudinally incompressible enough to set the first anchor in the septal wall. In some embodiments, inner catheter 26 may also function as a holding tube for holding and rotating first anchor 16.

Holding tube 28 comprises the same or a different biocompatible material from that used to form outer catheter 22. Holding tube 28 must be flexible enough to be delivered through vasculature to the treatment area while still longitudinally incompressible enough to hold and/or push second anchor 18.

To ensure proper positioning, it is desirable that tension device 12 be visible using fluoroscopy, echocardiography, intravascular ultrasound, angioscopy, or another means of visualization. Where fluoroscopy is utilized, any or all of tension device 12 may be coated with a radiopaque material, or a radiopaque marker may be included on any portion of the device that would be useful to visualize.

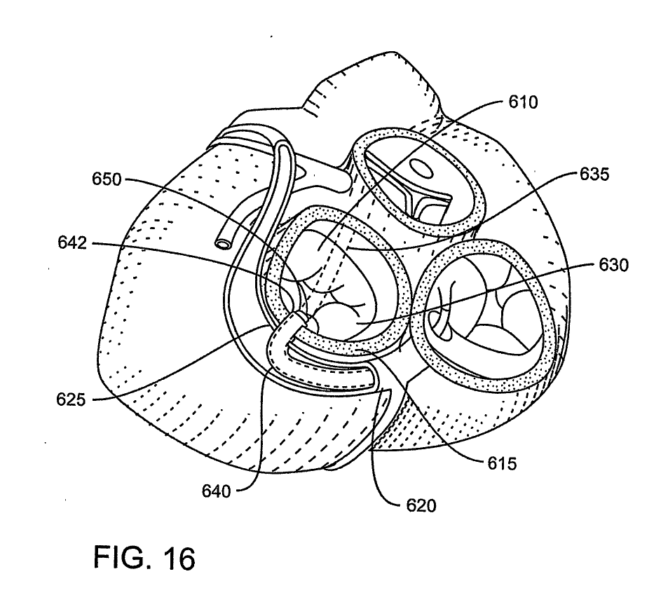

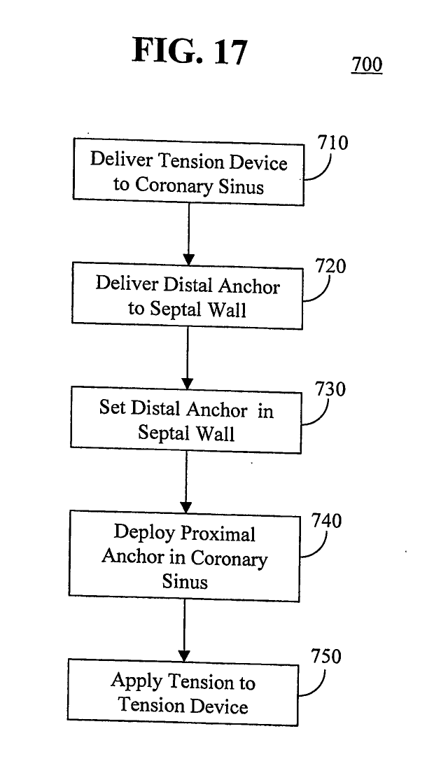

Another aspect of the present invention is a method for treating a dilated heart valve by affecting a mitral valve annulus. FIG. 16, which shows a system for treating a dilated heart valve at an intermediate step of the method, is used throughout the following discussion as a reference for the structures of the heart. FIG. 17 shows a flow diagram of one embodiment of the method 700 for treating a dilated heart valve, in accordance with the present invention. FIGS. 16 and 17 describe a method 700 of treating the mitral valve, those with skill in the art will readily recognize that the method and system may be modified to treat other cardiac valves. Additionally, although devices of the invention are shown and described as being routed through the coronary sinus, which is a vein, and being disposed across the left atrium, it will be appreciated that other coronary veins or arteries can be used to gain access to the atria or ventricles of the heart.

A system for treating mitral valve regurgitation is delivered to a position within the coronary sinus (Block 710). In the present embodiment, the system is system 10, as described above in FIG. 1.

For delivery, system 10 is in the configuration shown in FIG. 1. Tension device 12 is slidably received within delivery device 14. First anchor 16 is positioned within lumen 25 of delivery catheter 24 and second anchor 18 is positioned within lumen 27 of inner catheter 26. Push rod 28 abuts the proximal end of second anchor 18. Inner catheter 26 abuts the proximal end of first anchor 16.

Prior to delivery of tension device 12, a puncturing device is delivered to the coronary sinus to puncture a hole through the coronary sinus wall 625 and the heart wall 615 to gain access to the left atrium. Ideally, the hole is located adjacent the posterior leaflet 630 of mitral valve 610. The puncturing device may be a hollow needle radially extended from a side lumen of a puncture catheter. A guidewire may be advanced through the vasculature, the puncture device and the hollow needle to exit into the left atrium. The guidewire provides a pathway to the left atrium for subsequent insertions of catheters and other devices. In one embodiment, the puncture catheter is removed and a dilating catheter is advanced to the coronary sinus over the guidewire. The dilating catheter may be used to create a larger opening in the coronary sinus wall and the heart wall for insertion of delivery device 14 into the left atrium.

Delivery device 14 carrying tension device 12 is passed through the venous system and into a patient's coronary sinus and left atrium. This may be accomplished by inserting delivery device 14 into a femoral vein, through the inferior vena cava, and into coronary sinus 620. Alternative pathways to the coronary sinus may be used and are known to those with skill in the art. The procedure may be visualized using fluoroscopy, echocardiography, intravascular ultrasound, angioscopy, or other means of visualization.

The delivery device is advanced over the guidewire (not shown) until distal tip 642 of outer catheter 640 enters the left atrium. The first anchor is then delivered (Block 720) as follows. Delivery catheter 650 is advanced until the distal tip of delivery catheter 650 is adjacent the septal wall. Delivery catheter 650 may follow the pathway 635 illustrated as a dotted line in FIG. 16. Then, using inner catheter 26 as a push rod, first anchor 16 is set within the septal wall (Block 730). Delivery catheter 650 and inner catheter 26 are retracted leaving first anchor set within the septal wall. In alternative embodiments, inner catheter is rotated to insert a spiral anchor into the septal wall.

The second anchor is then deployed within the coronary sinus (Block 740). Continued retraction of delivery catheter 650 and inner catheter 26 deploys second anchor 18 within coronary sinus 620. Using second anchor 100 as described in FIGS. 2-6 as an example, removal of delivery catheter 650 and inner catheter 26 would deploy anchor 100 in its delivery state. Retraction of holding tube 42 releases anchor 100, allowing anchor 100 to unfold into the treatment state.

Tension is then applied to tension device 12 (Block 750). The practitioner exerts tension on tension device 12 by pulling on tether 20 via filament 42. Next, locking mechanism 30 is adjusted to maintain the desired tension. Locking mechanism 30 may be any of those described above or any device that will maintain the desired tension on tether 20. Once the tension device is locked in place the practitioner may remove filament 42 and outer catheter 22.

Variations of the device and methods described above will be apparent to those of ordinary skill in the art. For example, the system 10 may be configured to transect multiple chambers of the heart and apply tension across multiple valves.

Variations and alterations in the design, manufacture and use of the system and method are apparent to one skilled in the art, and may be made without departing from the spirit and scope of the present invention. While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A system for treating a dilated cardiac valve, comprising:
a delivery device including an inner catheter received in an outer catheter; and
a tension device slidably disposed within the inner catheter, the tension device including a first anchor connected to a second anchor by a tension member, the second anchor including a portion complementary to the wall of a cardiac vessel, wherein, when the tension device is delivered proximate the cardiac valve, the first anchor is inserted into a first cardiac wall and the second anchor expands within the cardiac vessel to apply tension across heart chamber via the tension member and the first anchor to reduce an annulus of the dilated cardiac valve.

2. The system of claim 1 wherein the first anchor is selected from the group consisting of a coil barbed anchor, a hooked anchor and a harpoon barbed anchor.

3. The system of claim 2 wherein the first anchor is rotatable to facilitate controlled insertion into the first cardiac wall.

4. The system of claim 1 wherein the first cardiac wall comprises an annulus of the dilated valve.

5. The system of claim 1 wherein the second anchor comprises a stent selected from the group consisting of a self-expanding stent and a balloon-expanding stent.

6. The system of claim 1 wherein the second anchor comprises an arcuate tubular body having an arc that complements a curve of at least a portion of the cardiac vessel.

7. The system of claim 1 wherein the second anchor comprises a tubular body having a first portion hingedly attached to a second portion.

8. The system of claim 1 wherein the cardiac vessel is the coronary sinus.

9. The system of claim 1 wherein the first anchor comprises a material selected from the group consisting of: stainless steel, nitinol, cobalt-based alloy, platinum, titanium, a thermoset plastic, a biocompatible alloy, a biocompatible material, and a combination thereof.

10. The system of claim 1 wherein the tension member comprises a material selected from the group consisting of: a thin wire or rod of stainless steel, nitinol, another flexible and strong material, rayon, nylon, polyester, or other similar material, and a combination thereof.

11. The system of claim 1 wherein the second anchor comprises a material selected from: flexible stainless steel, nitinol, cobalt-based alloy, biocompatible durable shape-memory polymers, and a combination thereof.

12. The system of claim 1 wherein the Inner catheter is a pushrod.

13. The system of claim 1 wherein the tension device further comprises a locking mechanism.

14. The system of claim 13 wherein the locking mechanism comprises a plurality of locking members disposed on the tension member.

15. A device for treating a dilated heart valve, the device comprising:
a first anchor disposed on a first end of a tension member; and
a second anchor slidably mounted on a second end of the tension member, the second anchor comprising an arcuate tubular body having an arc that complements a curve of at least a portion of a cardiac vessel adjacent the dilated valve.

16. The device of claim 15 wherein the first anchor is selected from the group consisting of a coil barbed anchor, a hooked anchor and a harpoon barbed anchor.

17. The system of claim 15 wherein the first anchor is rotatable to facilitate controlled insertion into the first cardiac wall.

18. The system of claim 15 wherein the second anchor comprises a stent selected from the group consisting of a self-expanding stent and a balloon-expanding stent.

19. The system of claim 15 wherein the second anchor comprises a tubular body having a first portion hingedly attached to a second portion.

20. A system for treating a dilated heart valve comprising:
means for inserting a first anchor into a first atrial wall proximate the dilated heart valve;
means for connecting the first anchor to a second anchor;
means for disposing the second anchor within a cardiac vessel proximate the dilated heart valve; and
means for applying tension across the connecting means.

21. The system of claim 20 further comprising:
means for locking the means for applying tension.

22. The system of claim 20 wherein the dilated heart valve is a dilated mitral valve.

23. A method of treating a dilated heart valve, the method comprising:
delivering a tension device comprising a first anchor connected to a second anchor by a tension member to a location within a cardiac vessel proximate the dilated heart valve;
inserting the first anchor into a heart wall proximate the dilated heart valve;
positioning the second anchor upon a cardiac vessel wall opposite the heart wall, wherein an arcuate portion of the second anchor is complementary to the cardiac vessel wall;
reducing an annulus of the dilated heart valve via the tension device.

24. The method of claim 23, wherein delivering the tension device comprises inserting the tension device within a catheter; and
delivering the catheter and the tension device to a location within the cardiac vessel proximate the dilated heart valve.

25. The method of claim 24 wherein delivering the catheter and tension device comprises:
positioning the catheter adjacent the cardiac vessel wall to insert the first anchor through the cardiac vessel wall;
pushing the first anchor through a heart chamber and into the heart wall opposite the cardiac vessel with an inner catheter;
retracting the catheter to release the second anchor within the cardiac vessel.

26. The method of claim 23 wherein the dilated heart valve is the mitral valve.

27. The method of claim 23 wherein positioning the second anchor comprises cinching a locking mechanism along a portion of the tension member proximal the second anchor to adjust a length of the tension member.
